(19) United States
(12) Patent Application Publication
(10) Pub. No.: US 2020/0140225 A1
(43) Pub. Date: May 7, 2020

(54) LINE HANDLING SYSTEM

(71) Applicant: ViewRay Technologies, Inc., Oakwood Village, OH (US)

(72) Inventors: Thomas CHMIELEWSKI, Aurora, OH (US); Gerald E. FOUGHT, Columbia Station, OH (US)

(73) Assignee: ViewRay Technologies, Inc., Oakwood Village, OH (US)

(21) Appl. No.: 16/666,064

(22) Filed: Oct. 28, 2019

Related U.S. Application Data
(60) Provisional application No. 62/755,992, filed on Nov. 5, 2018.

(51) Int. Cl. B65H 59/22

(52) U.S. Cl. CPC B65H 59/22; A61N 5/1081

(57) ABSTRACT

Systems for guiding line(s) including blocks through which the line(s) run. The blocks can include inner and outer guides with line running through both the inner and outer guides. The guides may include rollers, and an inner guide may be at least partially nested within an outer guide. The systems may be configured so that at least one of the blocks can move when the line moves and so that the first block and second block create tension in the line.

### RELATED APPLICATION(S)

This application claims priority to and the benefit of U.S. Provisional Application No. 62/755,992, filed Nov. 5, 2018, titled “Line Handling System,” which is hereby incorporated by reference.

### BACKGROUND

Radiotherapy can involve delivering radiation (e.g., x-rays) or therapeutic particles (e.g., proton or electron beams) from various treatment angles. In some treatment systems, the treatment delivery device may move to different angles around a patient by being mounted on a rotatable gantry. Certain designs of these systems will require handling mechanisms for the cables, lines, etc., that will run from devices located on the gantry to locations off of the gantry. Such handling mechanisms may be employed, for example, in order to take up slack or to feed out a line in an orderly manner. Such devices may also “accumulate” line slack in an orderly fashion.

While the examples provided herein depict line handling in conjunction with a radiotherapy system, it is contemplated that the technologies of this disclosure may be implemented in other instances or fields where the handling of lines is required. For example, the disclosed technologies can be implemented in industrial, manufacturing, robotics, assembly, or other suitable applications where the technical benefits of the disclosed subject matter can be realized.

### SUMMARY

Line handling systems designs are disclosed that may be used in applications where the accumulation and/or delivery of line(s) are required. Certain implementations may include one or more blocks having inner and outer guides with line running through the inner and outer guides. The guides can optionally include rollers. In certain embodiments, an inner guide may be at least partially nested within an outer guide and may be, for example, at least partially concentric with the outer guide.

In certain implementations, the system may be designed so that at least one of the blocks will move when the line moves. For example, a two block system can have both of the blocks move or can have one block fixed so as not to move when the line moves through it. Blocks utilized herein can also be configured to create tension in a line. For example, tension may be created at least partially by gravity acting on the mass of the block.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating a simplified perspective view of an exemplary line handler and gantry in accordance with certain aspects of the present disclosure.

FIG. 2 is a diagram illustrating simplified elevational views of an exemplary line handler in an unspooled configuration and in a spooled configuration in accordance with certain aspects of the present disclosure,

FIG. 3 is a diagram illustrating a simplified cross-sectional view of an exemplary line handler in accordance with certain aspects of the present disclosure,

FIG. 4 is a diagram illustrating a simplified cross-sectional view of rollers in a first block of an exemplary line handler in accordance with certain aspects of the present disclosure.

FIG. 5 is a diagram illustrating a simplified cross-sectional view of rollers in a second block of an exemplary line handler in accordance with certain aspects of the present disclosure.

### DETAILED DESCRIPTION

The present disclosure contemplates systems and methods for line handling allowing, for example, the reliable spooling of a line or lines (for example, water lines, gas lines, power cables, signal/data cables, rope, etc.). While the technologies herein are described with reference to use with a rotating gantry, they can be employed with any other device that would benefit from the orderly delivery or take-up of a line during operation. They may also be employed in a system where the line(s) are merely manipulated by humans.

To allow for the reliable and rapid delivery or accumulation of a line, the disclosed systems can be configured to keep a line under tension such that it will not pile up, tangle, or exhibit movement other than that required during operation of the system with which it is used.

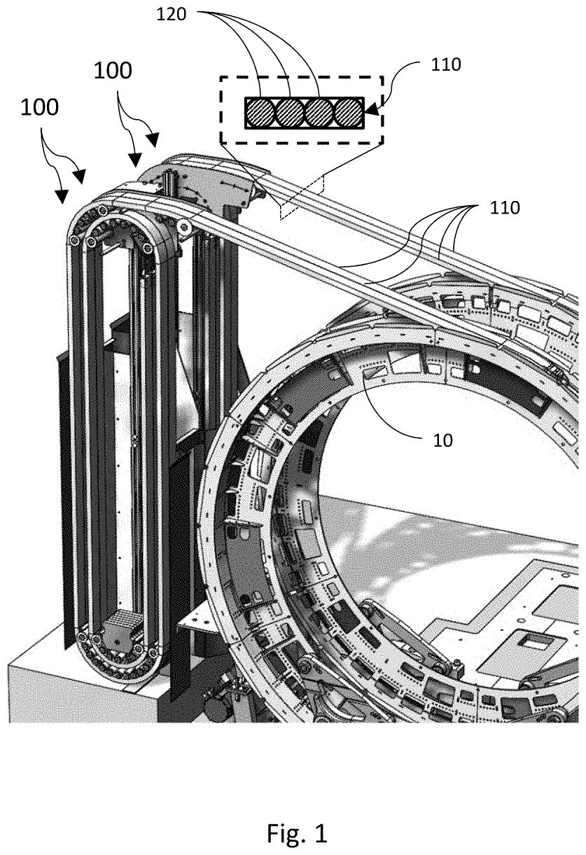

FIG. 1 is a diagram illustrating a simplified perspective view of an exemplary line handler and gantry in accordance with certain aspects of the present disclosure. The system illustrated in FIG. 1 actually depicts four similar line handlers 100 (two pairs of two) that are coupled to gantry 10, to allow the take-up of four lines 110 that may, for example, be used to power equipment mounted on gantry 10.

As seen in FIG. 1, line handler 100 and gantry 10 are connected by line 110, such that when gantry 10 rotates clockwise, line 110 is unspooled from line handler 100 and wraps around gantry 10. Conversely, when gantry 10 rotates counterclockwise, line 110 is spooled by line handler 100. As used herein, the terms “spooled” and “unspooled” refer respectively to the taking up and dispensing of any of lines 110 described herein.

While the illustration of FIG. 1 depicts line 110 as an elongated strip, it is contemplated that lines used with the technologies of the present disclosure may have alternative shapes or cross-sections. For example, line 110 may be a simple rope, may be a cable with square cross-section, etc. In addition, line 110 may be an assembly that can include a plurality of lines (or sub-lines). In one particular example, line 110 can be an assembly of multiple lines/sub-lines 120 disposed in a horizontal configuration, as shown in the expanded view within FIG. 1. The sub-lines can be, for example, power cables, cooling cables, data cables, etc.

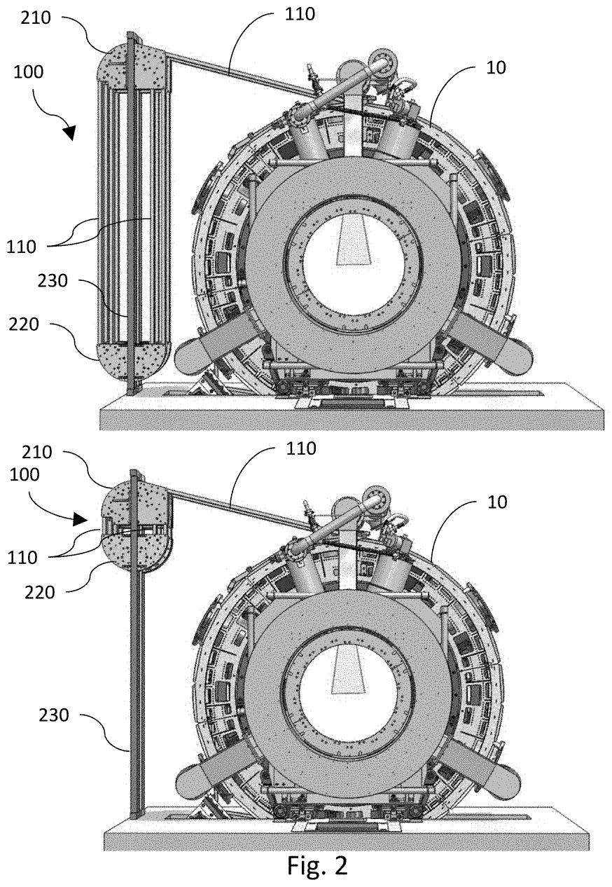

The top portion of FIG. 2 shows an exemplary line handler 100 in a spooled/accumulated configuration (i.e., a greater portion of line 110 is wrapped up by line handler 100). The bottom portion of FIG. 2 shows a state where gantry 10 has rotated clockwise, line 110 has unspooled, and a greater portion of line 110 is wrapped around gantry 10.

In an exemplary system for guiding a line (e.g., line handler 100), line 110 can be spooled between a first block 210 and a second block 220. The term “block,” as used herein, refers generally to an assembly through which a line may pass. In some implementations, a block can include a plurality of guides so that a line may pass through the block more than once.

Line handler 100 may be configured so that at least one of the first block or the second block moves when line 110 moves. In one example, first block 210 and second block 220 can both be configured to move during operation (e.g., toward and away from one another). In the particular example depicted in FIG. 2, first block 210 is fixed so as not to move when line 110 moves.

In some implementations, first block 210 and second block 220 can be supported by and operatively connected by a track 230, such that block movement occurs along track 230 when line 110 is spooled or unspooled. In the example depicted in FIG. 2, block 220 moves vertically along track 230 toward block 210 when line 110 is unspooled.

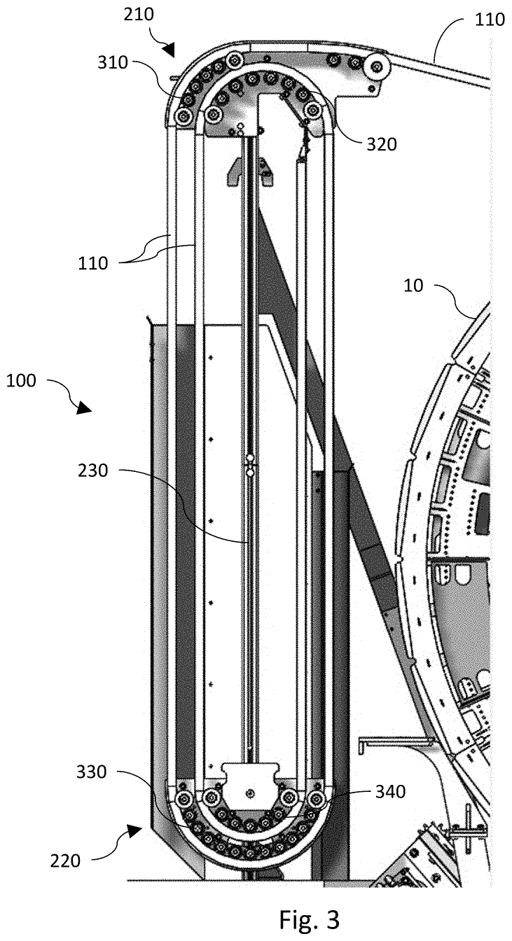

FIG. 3 is a diagram illustrating a simplified cross-sectional view of an exemplary line handler in accordance with certain aspects of the present disclosure. In the cross-section shown in FIG. 3, line 110 extends into line handler 100 and is wrapped in spiral-like layers within line handler 100 between first block 210 and second block 220. In the implementation of FIG. 3, first block 210 can include a first outer guide 310 and a first inner guide 320, with line 110 running through first outer guide 310 and first inner guide 320. Also, second block 220 can include a second outer guide 330 and a second inner guide 340, with line 110 running through second outer guide 330 and second inner guide 340.

As used herein, the term “guide” refers to things that can guide the movement of a line, for example, wheels, pulleys, rollers, gears, movable tracks, stationary tracks, etc. Also, while some guides are referred to herein as “inner” guides and “outer” guides, the terms “inner” and “outer” are not necessarily meant to limit a particular guide to being an innermost or outermost guide, but rather are used for descriptive purposes to merely indicate that one guide is located at a smaller radius than the other. This is because some implementations can include three or more guides in a block to increase the amount of line 110 that can be accumulated by line handler 100. In such a case, the guides could also be referred to as a first guide, second guide, third guide, etc. The present disclosure contemplates that each of the blocks may contain two or more guides. Finally, when referring to the number of guides within a block, it is understood that a guide such as guide 310 in FIG. 3 is considered a guide, even though it does not necessarily guide the path of a line for a full 180 degrees as many guides will.

Some or all of the guides within a block can be oriented in a nested configuration. As shown in FIG. 3, first block 210 and second block 220 each include two guides. In some implementations, first inner guide 320 can be at least partially nested within the first outer guide 310. Similarly, second inner guide 340 can be at least partially nested within second outer guide 330 in certain implementations. In still other implementations, both first block 210 and second block 220 can have guides that are nested.

As used herein, the term “nested” refers to one guide being at least partially within a general region that makes up another guide. For example, referring to FIG. 3, second inner guide 340 is clearly nested within second outer guide 330. Similarly, first inner guide 320 is generally nested within first outer guide 310, even though the portion of first outer guide 310 closest to gantry 10 does not continue to wrap around first inner guide 320. Also, because there can be any number of guides, any combination and number of guides may have nested portions. For example there may be one, two, three, etc., guides in a particular block, with any number of such guides being nested to varying degrees.

In some implementations, first inner guide 320 may be at least partially concentric with first outer guide 310. In other implementations, second inner guide 340 can be concentric with second outer guide 330. As used herein, concentric means precisely, or nearly precisely, concentric as illustrated by second inner guide 340 and second outer guide 330. Other implementations, such as shown by first outer guide 310 and first inner guide 320, can have a portion of the two guides being concentric, while other portions are not. For example, as shown in FIG. 3, the upper left portion of first outer guide 310 is approximately concentric with first inner guide 320, while the upper right portion of first outer guide 310 extends in a more linear manner towards gantry 10.

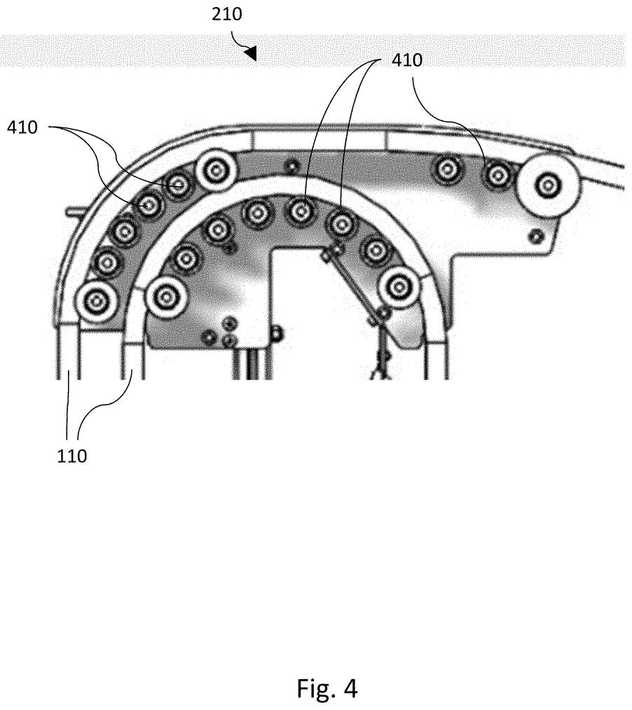

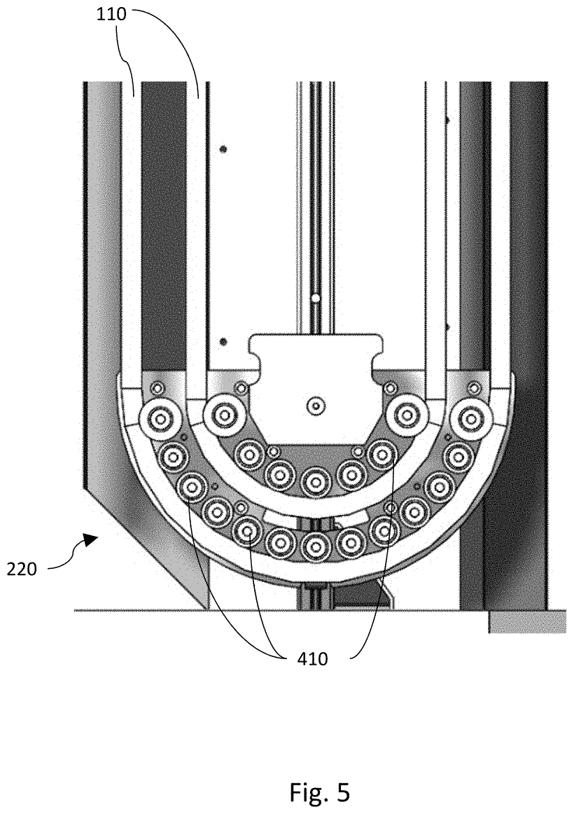

FIG. 4 is a diagram illustrating a simplified cross-sectional view of exemplary rollers in the first block of an exemplary line handler in accordance with certain aspects of the present disclosure. FIG. 5 is a diagram illustrating a simplified cross-sectional view of exemplary rollers in the second block of an exemplary line handler in accordance with certain aspects of the present disclosure.

As shown in both FIG. 4 and FIG. 5, the inner guides and/or the outer guides can include a number of rollers 410 that can support line 110, the outer envelope of such rollers creating a movement path for line 110. Rollers can, for example, allow line 110 to move within line handler 100 with reduced friction. In other implementations, instead of rollers, the guides may include a smooth track or channel over which line 110 may slide. Rollers, tracks, channels, and guides can define a bend radius for line 110. Certain types of lines, fiber optic cable for example, cannot (or should not) be bent in a smaller radius than a particular value. The arrangement of guides or rollers 410 can thus be chosen to coincide with any permissible bend radius of the line.

Some implementations of the present disclosure can include mechanisms that create a desired tension in line 110, for example, as needed to reduce binding or tangles in the line or to avoid excess slack. In some implementations, the first block and the second block can create tension in a line. For example, in the implementation of FIG. 2, the weight of second block 220 can create tension in line 110. Thus, in a configuration where first block 210 and second block 220 are oriented at least partially in a vertical direction, tension can be created at least partially by gravity operating on second block 220. In some implementations, second block 220 can also include a counterweight 510 to increase the amount of tension in line 110. Counterweight 510 can be a single mass, or as seen in FIG. 1, a combination of masses.

Other implementations of line handler 100 can include a spring mechanism or mechanisms that push or pull apart first block 210 and second block 220. In this way, tension can be created at least partially by the spring mechanism(s) to provide an alternative (or additional) tensive force other than gravity, as may be used in implementations where line handler 100 is in a horizontal, angled or even inverted (second block 220 above the first block 210) configuration. While a spring between the blocks is one potential implementation, when the term “spring mechanism” is used herein it contemplates any device, element, mechanism or mechanisms that push or pull apart the blocks. For example, a spring, multiple springs, elastic bands, actively controlled hydraulics, etc., may be used.

When the present disclosure refers to line handlers or portions thereof creating “tension” in a line, it should be noted that such element(s) need not be entirely responsible for the line tension. The element(s) referred to need be only partially responsible for tension in the line. For example, the second block 220 of FIG. 2 is partially responsible for creating tension in line 110, along with the force of gravity and any rotational resistance of gantry 10. Similarly, two blocks can be partially responsible for creating tension in the line, along with a spring that may be employed between them.

In the following, further features, characteristics, and exemplary technical solutions of the present disclosure will be described in terms of items that may be optionally claimed in any combination:

Item 1: A system for guiding a line, the system comprising: a first block comprising a first outer guide and a first inner guide, the line running through the first outer guide and the first inner guide; and a second block comprising a second outer guide and a second inner guide, the second inner guide at least partially nested within the second outer guide, the line running through the second outer guide and the second inner guide and, the system being configured so that at least one of the first block or second block moves when the line moves, and the first block and the second block create tension in the line.

Item 2: the system of item 1, wherein the inner guides comprise a plurality of rollers.

Item 3: the system of any one of the preceding items, wherein the outer guides comprise a plurality of rollers.

Item 4: the system of any one of the preceding items, wherein the first inner guide is at least partially concentric with the first outer guide.

Item 5: the system of any one of the preceding items, wherein the second inner guide is at least partially concentric with the second outer guide.

Item 6: the system of any one of the preceding items, wherein the first block is fixed so as not to move when the line moves.

Item 7: the system of any one of the preceding items, wherein the tension is created at least partially by gravity operating on the second block.

Item 8: the system of any one of the preceding items, the second block further comprising a counterweight to increase the tension in the line.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

What is claimed is:

1. A system for guiding a line, the system comprising:
a first block comprising a first outer guide and a first inner guide, the line running through the first outer guide and the first inner guide; and
a second block comprising a second outer guide and a second inner guide, the second inner guide at least partially nested within the second outer guide, the line running through the second outer guide and the second inner guide and, the system being configured so that at least one of the first block or second block moves when the line moves, and the first block and the second block create tension in the line.

2. The system of claim 1, wherein the inner guides comprise a plurality of rollers.

3. The system of claim 1, wherein the outer guides comprise a plurality of rollers.

4. The system of claim 1, wherein the first inner guide is at least partially concentric with the first outer guide.

5. The system of claim 1, wherein the second inner guide is at least partially concentric with the second outer guide.

6. The system of claim 1, wherein the first block is fixed so as not to move when the line moves.

7. The system of claim 6, wherein the tension is created at least partially by gravity operating on the second block.

8. The system of claim 7, the second block further comprising a counterweight to increase the tension in the line.

9. The system of claim 1, wherein the system further comprises a spring mechanism or mechanisms that push or pull apart the first block and the second block.

10. The system of claim 9, wherein the tension is created at least partially by the spring mechanism(s).

11. The system of claim 1, wherein the line is an assembly.

12. The system of claim 11, wherein the assembly comprises a plurality of sub-lines.

13. The system of claim 12, wherein the sub-lines are disposed in a horizontal configuration.
